(19) United States
(12) Patent Application Publication
(10) Pub. No.: US 2023/0255637 A1
(43) Pub. Date: Aug. 17, 2023

(54) MEDICAL CLIP

(71) Applicant: Aesculap AG, Tuttlingen (DE)

(72) Inventors: Thomas Scholten, Tuttlingen (DE); Jens Beger, Tuttlingen (DE); Thomas Pleil, Bad Duerrheim (DE); Alexander Happle, Huefingen (DE); Jan Reich, Tuningen (DE); Matthias Schweizer, Tuttlingen (DE); Thomas Hagen, Tuttlingen (DE); Natalia Dalmann, Tuttlingen (DE)

(21) Appl. No.: 18/138,795

(22) Filed: Apr. 25, 2023

Related U.S. Application Data
(63) Continuation of application No. PCT/EP2021/079946, filed on Oct. 28, 2021.

(30) Foreign Application Priority Data
Nov. 3, 2020 (DE) 10 2020 128 942.1

(51) Int. Cl. A61B 17/122

(52) U.S. Cl. CPC A61B 17/1227; A61B 2017/00526

(57) ABSTRACT

A medical clip includes a first clamping arm, a second clamping arm, and a biasing element with a first and a second end. The first clamping arm has a first clamping arm end, which is connected to the first end of the biasing element by way of a first connecting portion. The second clamping arm has a second clamping arm end, which is connected to the second end of the biasing element by way of a second connecting portion. The clip includes a box lock with a connection perforation arranged or formed on the first connecting portion and a connection portion comprised by the second connecting portion and passing through the connection perforation. The first clamping arm and the second clamping arm are maximally proximate to one another in a basic position.

### CROSS-REFERENCE TO RELATED APPLICATIONS

This application is a continuation of International Application No. PCT/EP2021/079946 filed on Oct. 28, 2021, which claims priority to German Application No. 10 2020 128 942.1 filed on Nov. 3, 2020. The contents of International Application No. PCT/EP2021/079946 and German Application No. 10 2020 128 942.1 are incorporated herein by reference in their entireties and for all purposes.

### FIELD

The present disclosure relates to medical clips generally, and more specifically to a medical clip, in particular in the form of an aneurysm clip, which comprises a first clamping arm, a second clamping arm, and a biasing element with a first and a second end, wherein the first clamping arm has a first clamping arm end, which is connected to the first end of the biasing element by way of a first connecting portion, wherein the second clamping arm has a second clamping arm end, which is connected to the second end of the biasing element by way of a second connecting portion, wherein the clip comprises a box lock with a connection perforation arranged or formed on the first connecting portion and a connection portion comprised by the second connecting portion and passing through the connection perforation, wherein the first clamping arm and the second clamping arm are maximally proximate to one another, in particular abutting against one another, in a basic position of the clip, and can be moved away from one another against the action of the biasing element from the basic position into an opening position, wherein the first clamping arm commencing from the first clamping arm end and the second clamping arm commencing from the second clamping arm end are of curved or angled configuration in the direction toward free ends thereof, and wherein two female delimiting surfaces facing toward one another are formed on the connection perforation and two male delimiting surfaces facing away from one another and toward the female delimiting surfaces are formed on the connection portion.

### BACKGROUND

Medical clips of the kind described at the outset are known in a wide variety of ways. In particular, they are used as aneurysm clips. For such clips, it is important that the biasing force exerted by the biasing element, thus in particular a spring force, has a predefined value. To determine the spring force of aneurysm clips, they are opened to a certain value in accordance with an existing standard and placed on a test device. The force determined in this process, also known as the closing force, must then be within a certain tolerance window specified by the standard. Otherwise, the clip must be revised or discarded as unusable.

However, one problem is that a portion of the permissible tolerance is already consumed by the measurement inaccuracy of the test device and by the test procedure itself. Another, not insignificant portion of the permissible tolerance is lost due to friction in the region of the box lock, so that in practice the tolerance window available for the test procedure is significantly smaller than the permissible range specified by the standard. In particular in the case of aneurysm clips, the clamping arms of which, also referred to as jaw parts, deviate from an elongate form, i.e. which, as described at the outset, commencing from the first clamping arm end and from the second clamping arm end are curved, for example in an arcuate manner, or are angled, having a single bend or multiple bends, in the direction toward free ends of the clamping arms, due to the action of the biasing element, torsional forces occur in a connection region of the clip, i.e. in that region in which the box lock is arranged or formed, in particular when closing the clip, i.e. when moving the clamping arms from an opened position toward one another. These torsional forces twist the connecting portions until the male and female delimiting surfaces come into contact on both sides. In the case of known clips, this is accompanied by a jamming of the box lock. This jamming can cause such high friction that in the worst case the tolerance window permitted for testing the spring force is completely used up. One disadvantage here is, in particular, that clips complying with or not complying with the test standard can no longer be clearly distinguished.

### SUMMARY

In a first aspect, a medical clip, in particular in the form of an aneurysm clip, comprises a first clamping arm, a second clamping arm, and a biasing element with a first and a second end. The first clamping arm has a first clamping arm end, which is connected to the first end of the biasing element by way of a first connecting portion. The second clamping arm has a second clamping arm end, which is connected to the second end of the biasing element by way of a second connecting portion. The clip comprises a box lock with a connection perforation arranged or formed on the first connecting portion and a connection portion comprised by the second connecting portion and passing through the connection perforation. The first clamping arm and the second clamping arm are maximally proximate to one another, in particular abutting against one another, in a basic position of the clip and can be moved away from one another against the action of the biasing element from the basic position into an opening position. The first clamping arm commencing from the first clamping arm end and the second clamping arm commencing from the second clamping arm end are of curved or angled configuration in the direction toward free ends thereof. Two female delimiting surfaces facing toward one another are formed at the connection perforation and two male delimiting surfaces facing away from one another and toward the female delimiting surfaces are formed on the connection portion. The two female delimiting surfaces are each provided with a female, in particular in each case only one single, torsional bevel and/or the two male delimiting surfaces are each provided with a male, in particular in each case only one single, torsional bevel.

### BRIEF DESCRIPTION OF THE DRAWING FIGURES

The foregoing summary and the following description may be better understood in conjunction with the drawing figures, of which:

FIG. 1 is schematic perspective total view of a first embodiment of a medical clip and a clip application instrument;

FIG. 2 is a schematic, perspective enlarged view of a distal end region of the clip application instrument with a medical clip accommodated therein in an opening position thereof;

FIG. 3 is a view of the arrangement from FIG. 2 in the direction of the arrow A;

FIG. 4 is a view similar to FIG. 3, but with a hollow organ clamped off;

FIG. 5 is a view of the arrangement from FIG. 4 in the direction of the arrow B;

FIG. 6 is a perspective total view of a further embodiment of a medical clip, which has only male torsional bevels;

FIG. 7 is an enlarged partial view of the region C from FIG. 6;

FIG. 8 is a schematic section view along line 8-8 in FIG. 7 without acting torsional forces;

FIG. 9 is a schematic view of the arrangement similar to FIG. 8 with acting torsional forces;

FIG. 10 is a schematic perspective view of a further embodiment of a medical clip similar to FIG. 6;

FIG. 11 is a section view similar to FIG. 8 in the region of the box lock of the embodiment of the medical clip from FIG. 10;

FIG. 12 is a view similar to FIG. 11, but with torsional forces acting on the clamping arms;

FIG. 13 is a schematic partial view of a further embodiment of a medical clip in the region of the box lock with exclusively female torsional bevels;

FIG. 14 is a section view along line 14-14 in FIG. 13;

FIG. 15 is a schematic view similar to FIG. 14 but with acting torsional forces;

FIG. 16 is a schematic, partially broken side view of a further embodiment of a medical clip in the region of the box lock with male and female torsional bevels;

FIG. 17 is a schematic section view along line 17-17 in FIG. 16 without acting torsional forces;

FIG. 18 is a schematic view similar to FIG. 17 but with acting torsional forces;

FIG. 19 is a schematic view of a further embodiment of a medical clip; and

FIG. 20 is a schematic view of a further embodiment of a medical clip.

### DETAILED DESCRIPTION

Although the present disclosure is illustrated and described herein with reference to specific embodiments, the present disclosure is not intended to be limited to the details shown. Rather, various modifications may be made in the details without departing from the present disclosure.

The present disclosure relates to a medical clip, in particular in the form of an aneurysm clip, which comprises a first clamping arm, a second clamping arm, and a biasing element with a first and a second end, wherein the first clamping arm has a first clamping arm end, which is connected to the first end of the biasing element by way of a first connecting portion, wherein the second clamping arm has a second clamping arm end, which is connected to the second end of the biasing element by way of a second connecting portion, wherein the clip comprises a box lock with a connection perforation arranged or formed on the first connecting portion and a connection portion comprised by the second connecting portion and passing through the connection perforation, wherein the first clamping arm and the second clamping arm are maximally proximate to one another, in particular abutting against one another, in a basic position of the clip and can be moved away from one another against the action of the biasing element from the basic position into an opening position, wherein the first clamping arm commencing from the first clamping arm end and the second clamping arm commencing from the second clamping arm end are of curved or angled configuration in the direction toward free ends thereof, and wherein two female delimiting surfaces facing toward one another are formed at the connection perforation and two male delimiting surfaces facing away from one another and toward the female delimiting surfaces are formed on the connection portion, wherein the two female delimiting surfaces are each provided with a female, in particular in each case only one single, torsional bevel and/or wherein the two male delimiting surfaces are each provided with a male, in particular in each case only one single, torsional bevel.

The proposed further development enables, in particular, a rotation of the connecting portions in the region of the box lock, so that the clamping arms can twist freely or nearly freely. A collision on both sides of the male and female delimiting surfaces facing toward one another in the region of the box lock can be ruled out due to the torsional bevels. In contrast to conventional clips which do not have torsional bevels, the proposed female and/or male torsional bevels formed on the female delimiting surfaces and/or on the male delimiting surfaces make it possible to replace a point contact of the connecting portions in the region of the box lock in conventional clips as a result of a torsion of the clamping arms, namely when edges on the connection perforation and the connection portion meet one another, with a line contact due to the proposed torsional bevels. Instead of an edge-to-edge contact, an edge-to-surface contact can be achieved, now producing a line contact instead of a point contact as a result of a torsion. Therefore, the risk of a jamming of the connecting portions in the region of the box lock as a result of a torsion of the clamping arms can be significantly reduced. Concomitantly, friction in the region of the box lock as a result of a torsion of the clamping arms can also be reduced. In particular, the influence of the torsion of the clamping arms on the testing of the clamping force of the clip can thus be reduced. If both female and male torsional bevels are provided, an abutment of two surfaces in the region of the box lock upon a torsion of the connecting portions is made possible. Thus, in comparison to a formation of torsional bevels on one side, i.e. provided either only the female delimiting surfaces or only on the male delimiting surfaces, by beveling both the female and male delimiting surfaces in the connection region of the clip as proposed, i.e. by forming female and male torsional bevels, friction in the region of the box lock can be further reduced. Such a reduction in friction has a positive effect, in particular, on a scattering of measured values when determining the spring force of medical clips, which in turn can reduce waste during production. The male and/or the female torsional bevels are applied to precisely those edges of the connection perforation or the connection portion that pivot out upon a torsional load of the clamping arms and lead to a point or line contact. The male and/or the female torsional bevels are preferably formed only in those regions of the connection perforation and the connection portion in which the connecting portions are located while the maximum torsional moment is acting. These are, in particular, the positions starting from a completely closed medical clip, i.e. when the clamping arms abut against one another, up to an opening width of about 10% to about 50%, preferably up to an opening width of about 30%, based on a maximum possible opening of the clip.

It is favorable if the two female torsional bevels extend in parallel to one another and/or if the two male torsional bevels extend in parallel to one another. Torsional bevels of that kind can be produced in a simple manner, for example by milling. Furthermore, they ensure, in particular, optimal guidance of the connecting portions in the region of the box lock, since as a result of a torsion, the respective torsional bevels can always cooperate simultaneously with corresponding delimiting surfaces of the other connecting portion.

Preferably only the two female delimiting surfaces are provided with a female torsional bevel. This design makes it possible, in particular, to minimize friction in the region of the box lock and to still be able to counteract the opening of the clamping arms with a sufficient resistance.

Favorably only the two male delimiting surfaces are provided with a male torsional bevel. Here, too, friction can be reduced and at the same time it can be ensured that the opening of the clamping arms can be counteracted with sufficient resistance, i.e. they are not too yielding. The formation of male torsional bevels on the connection portion is also easier to produce than female torsional bevels in the region of the connection perforation.

The medical clip can be formed in a simple manner if the two male delimiting surfaces extend in parallel to one another. Such delimiting surfaces can, in particular in cooperation with female boundary surfaces that extend in parallel to one another, optimally guide an opening and closing movement of the medical clip. Here, it is advantageous, in particular, if play in the region of the box lock, i.e. in particular a spacing of the cooperating male and female delimiting surfaces, is minimal. In particular, the box lock should be designed in such a way that a free rotation of the connecting portions in the region of the box lock is not possible, if possible.

The medical clip can be produced in a simple manner if the connection portion is of elongated cuboidal configuration. Such a connection portion has, in particular, four connection portion edges running in parallel to a longitudinal axis defined by the connection portion. These can cooperate with connection portion edges of the connection perforation upon a torsion of the connecting portions. Furthermore, the connection portion edges can be beveled in a simple manner.

The box lock can be realized in a simple manner, in particular, by the connection perforation being of elongated hole-like configuration and defining a connection plane and by the two female delimiting surfaces extending in parallel to the connection plane. In particular, such a design is easy to manufacture. Furthermore, a defined pivoting of the clamping arms relative to one another can thus be predetermined by the connection plane defined in this way.

It is advantageous if the two male delimiting surfaces in the basic position extend in parallel or substantially in parallel to the connection plane. In other words, they can be oriented, in particular, in the basic position in parallel to female delimiting surfaces extending in parallel to one another. A pivot movement of the clamping arms in a defined pivot plane that extends in parallel to the connection plane can thus be predetermined.

According to a further preferred embodiment, provision may be made that the first clamping arm and the second clamping arm are curved out of the connection plane or angled relative to it in such a way that free ends thereof point in a direction that runs transversely, in particular perpendicularly, to the connection plane. Configuring clamping arms in that way makes it possible, in particular to be able to place the clip in a wide range of applications. In particular, in a minimally invasive application, for example, aneurysms can be clamped off that do not form a sacculation transverse, in particular perpendicular, to a longitudinal axis of the application instrument, but parallel or substantially parallel to the longitudinal axis of the instrument, also referred to as the application axis. A curvature of the clamping arms, in particular, may be continuous, such that a jaw part defined by the two clamping arms may, for example, be of arcuate configuration.

A particularly flexible application of medical clips can be achieved, in particular, by the first clamping arm and the second clamping arm being continuously curved. For example, a radius of curvature of the two clamping arms may be constant. Thus, in particular, an arcuate clip can be formed.

It is advantageous if the first clamping arm and the second clamping arm each have at least one bend, and if the at least one bend is arranged or formed in each case between a free end and the first and second clamping arm ends. With clips of that kind, for example, a hollow organ, in particular a blood vessel or a sacculation thereof, can be clamped off in a defined manner on the distal side of the bend. For example, a bend can be used to create an L-shaped jaw part defined by the two clamping arms. With two bends, in particular, a bayonet-shaped or double-L-shaped jaw part can be realized.

The first clamping arm and the second clamping arm in the basic position, in particular when they abut against one another, preferably extend in parallel to one another over their entire length. Such a configuration makes it possible, in particular, to reduce the risk of so-called “scissoring”, that is, the slipping of lateral edges of the clamping arms against one another, which can lead to injury to tissue accommodated between the clamping arms.

It is favorable if the first clamping arm and the second clamping arm are pivotable relative to one another about a pivot axis from the basic position into an abutment position in which the clamping arms are further apart from one another relative to the basic position. Such a design enables, in particular, a simple handling of the medical clip, for example with an application instrument configured for this purpose, as well as a defined opening and closing thereof by moving the clamping arms away from one another and back toward one another.

The pivot axis preferably extends transversely, in particular perpendicularly, to the connection plane. This design can, in particular, minimize the risk of “scissoring” described above. In addition, a defined handling of the medical clip, in particular an application thereof, is possible for a user. For this purpose, in particular, the application instrument may be configured in such a way that a surgeon actuates actuating elements of the application instrument arranged so as to be movable relative to one another in a plane, wherein the plane extends in parallel to the connection plane.

In order to be able to ensure a defined guidance of the connecting portions in the region of the box lock, it is advantageous if between about 40% and 60% of the two female delimiting surfaces, in particular between about 45% and about 55%, further in particular about 50%, are removed by the female torsional bevels.

Furthermore, it is advantageous if between about 40% and about 60% of the two male delimiting surfaces, in particular between about 45% and about 55%, further in particular about 50%, are removed by the male torsional bevels. Thus, in particular, an optimal guidance of the connecting portions on one another in the region of the box lock as a result of a torsion of the clamping arms can be ensured.

The medical clip can be formed in a simple manner if the female and/or the male torsional bevels define planar bevel surfaces. Optionally, the torsion surfaces, i.e. male and/or female, can be designed to extend into the respective delimiting surface without edges. Thus, in particular, clips can be opened and closed in a sensitive manner.

In accordance with a further preferred embodiment, provision may be made that the female delimiting surfaces and female bevel surfaces, which are defined by the female torsional bevels, enclose a female bevel angle and that the female bevel angle has a value in a range of about 5° to about 25°. In particular, the female bevel angle may have a value in a range of about 10° to about 18°, in particular the value may be about 15°. Providing the female bevel angle in the specified ranges makes it possible, in particular, to limit the configuration of the torsional bevels to the most unfavorable possible torsion of the clamping arms relative to one another. In particular, the female bevel angle can also limit a torsion of the clamping arms relative to one another.

Furthermore, it is advantageous if the male delimiting surfaces and male bevel surfaces, which are defined by the male torsional bevels, enclose a male bevel angle and if the male bevel angle has a value in a range of about 5° to about 25°, in particular a value in a range of about 10° to about 18°. Preferably, the male bevel angle has a value of about 15°. Here, too, the male bevel angle can be limited to the most unfavorable possible torsion or twisting of the clamping arms relative to one another. The selected male bevel angle can also limit, in particular, a torsion of the clamping arms relative to one another, i.e. basically define a stop.

Friction in the region of the box lock can be minimized in a simple manner by the female bevel angle corresponding or substantially corresponding to the male bevel angle.

It is favorable if the female and/or the male torsional bevels define curved bevel surfaces. In this way, an optimal guidance of the cooperating delimiting surfaces and bevel surfaces can be achieved.

It is advantageous if the curved bevel surfaces define intersection lines with an intersection plane at the respective connecting portion transverse to the delimiting surfaces thereof and if the intersection lines have a constant or increasing curvature commencing from the beveled delimiting surface. Curved bevel surfaces with a constant curvature can be defined, in particular, by surfaces of straight circular cylinders or straight hollow cylinders. For example, curved bevel surfaces with increasing curvature can define intersection lines that form the section of an ellipse.

Advantageously, the intersection lines of the torsion bevels define a section of an ellipse. Such an intersection line may have, in particular, an increasing curvature commencing from the beveled delimiting surface. In particular, a continuous movement of the connecting portions relative to one another as a result of a torsion of the clamping arms can be predetermined.

In order to minimize jamming of edges of cooperating male and female delimiting surfaces, it is advantageous if the connection perforation defines four connection perforation edges adjoining the female delimiting surfaces and if two connection perforation edges that are rotated against one another by 180° relative to a first longitudinal axis defined by the first connecting portion are beveled by the female torsion bevels. The female torsion bevels that are configured in the proposed manner thus enable a torsion of the connecting portions relative to one another, namely in such a way that instead of the point contact that occurs with conventional clips, which would result in jamming with very high friction, a line contact is achieved. Thus, in particular, a torsion of the connecting portions relative to one another can be made possible in a certain extent, wherein in the worst case, there is a line contact between cooperating surfaces or edges, which, compared to conventional clips, help to minimize friction in the region of the box lock.

Furthermore, it is favorable if the connection portion defines four connection portion edges and if two connection portion edges that are rotated against one another relative to a second longitudinal axis defined by the second connecting portion are beveled by the male torsional bevels. By beveling the male delimiting surfaces of the connection portion in the proposed manner, a defined deflection of the connecting portions relative to one another as a result of a torsion of the clamping portions is made possible. In contrast to conventional clips, this prevents a point contact between cooperating edges of the male and female delimiting surfaces, and instead enables a line contact between cooperating edges and surfaces of the two connecting portions. This can prevent friction in the region of the box lock as a result of torsional forces acting on the clamping arms of the clip.

In accordance with a further preferred embodiment, provision may be made that the two female torsional bevels and/or the two male torsional bevels are configured to guide and to delimit a torsional movement of the connecting portions relative to one another about longitudinal axes of the respective connecting portions upon closing the clamping arms when an object to be clamped is accommodated between them. In particular, torsions of the connecting portions in the region of the box lock can thus be made possible as a result of torsional forces acting on the clamping arms upon closing the clamping arms when an object to be clamped is accommodated between them.

It is advantageous if the two male delimiting surfaces and the two female delimiting surfaces each define sections of outer surfaces of a straight circular cylinder and if connecting portion longitudinal axes of the two connecting portions each define circular cylinder longitudinal axes of the respective outer surfaces. In particular, the male and/or female torsional bevels may be of curved configuration and also define sections of outer surfaces of straight circular cylinders. Such a configuration can be achieved, in particular, if the connection portion has a circular cross section and if portions of the first connecting portion that delimit the connection perforation also have circular cross sections. In this way, when the clamping arms are pivoted relative to one another, even if torsional forces are acting on them, there is no reduction of a play between the connection perforation and the connection portion.

The medical clip can be formed in a simple manner if the outer surface radii of the male delimiting surfaces and the female delimiting surfaces are identical or substantially identical.

The medical clip can be formed in a simple manner, in particular, by the connecting portions, in particular the connection perforation and/or the connection portion, being formed by milling, by electrochemical metal erosion, by a generative manufacturing process, in particular 3D printing, or by sink erosion.

Further, it may be advantageous if male and/or female delimiting surfaces and/or the male and/or female torsional bevels are provided with a friction-reducing coating. In particular, possible friction losses in the region of the box lock can thus be further reduced. As an alternative to a friction-reducing coating, the medical clip, in particular the cooperating connecting portions in the region of the box lock, may be made of a material that forms a material pairing with favorable sliding properties.

It is favorable if the first clamping arm and/or the second clamping arm and/or the biasing element are formed by a generative manufacturing process. This makes it possible, in particular, to form the medical clip as a whole by a generative manufacturing process, for example, the entire clip can be produced by using a 3D printing process.

The first clamping arm and the second clamping arm are preferably biased against one another in the basic position. In particular, this design can help to ensure that an object to be clamped that is arranged between the clamping arms can be permanently held in a clamping manner and with a defined clamping force.

A biasing element can be configured in a simple manner in the form of a coil spring with at least one complete winding.

In order to avoid rejection reactions of the medical clip by the body of a patient, it is advantageous if the clip is made of at least one biocompatible material, in particular only of one single biocompatible material.

The biocompatible material is preferably a metal, in particular titanium, or a plastic. The stated materials can be used in a simple manner to form medical clips.

In order to be able to treat, in particular, sacculations on hollow organs in a defined and simple manner, it is advantageous if the clip is configured in the form of an aneurysm clip.

The clip is preferably of one-piece, in particular monolithic configuration. Such a clip can be formed, for example, by a generative manufacturing process or from a blank.

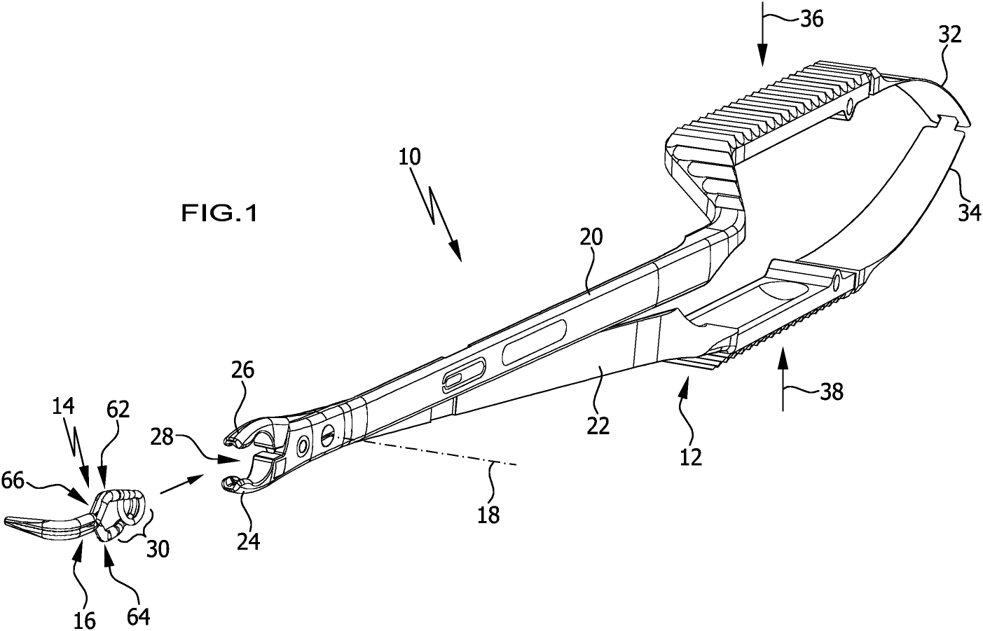

An embodiment of a clip application system is schematically depicted in FIG. 1 and is denoted as a whole with the reference numeral 10. It comprises an application instrument 12 and a first embodiment of a medical clip 14, which is configured in the form of an aneurysm clip 16.

The application instrument 12 comprises two branches 20 and 22 which can be pivoted relative to one another about a pivot axis 18 and at the distal ends of which tool elements 24 and 26 are formed, which form a receptacle 28 for accommodating a proximal end region 30 of the clip 14.

The two branches 20 and 22 are held in a basic position by two cooperating leaf spring elements 32 and 34, which are arranged or formed at proximal ends of the branches 20 and 22, in a position which is as far apart or is deflected as far as possible from one another. By applying an actuating force on the branches 20 and 22 toward one another, i.e. in the direction of the arrows 36 and 38, the two tool elements 24 and 26 are also moved toward one another. The clip 14 is thereby opened.

The medical clip 14 comprises a biasing element 40 with a first end 42 and a second end 44. In the embodiment of the medical clip 14 depicted in FIGS. 1 to 5, the biasing element 40 is configured in the form of a coil spring 46 that comprises more than one winding.

The clip 14 comprises a first clamping arm 48 and a second clamping arm 50.

The first clamping arm 48 extends from a free end 54 to a first clamping arm end 56. The second clamping arm 50 extends from a free end 58 to a second clamping arm end 60.

The first end 42 of the biasing element 40 is connected to the first clamping arm end 26 by way of a first connecting portion 62. The second end 44 of the biasing element 40 is connected to the second clamping arm end 60 of the second clamping arm 50 by way of a second connecting portion 64.

The medical clip 14 comprises a box lock 66, which comprises a connection perforation 68 and a connection portion 70 passing through the connection perforation 68. The connection perforation 68 is arranged or formed on the first connecting portion 62. The connection portion 70 is formed on the second connecting portion 64 and is thus comprised thereby.

The clamping arms 48 and 50 are maximally proximate to one another in a basic position of the clip 14. In the embodiment shown in FIGS. 1 to 5, they abut against one another in the basic position, as schematically depicted in FIG. 1.

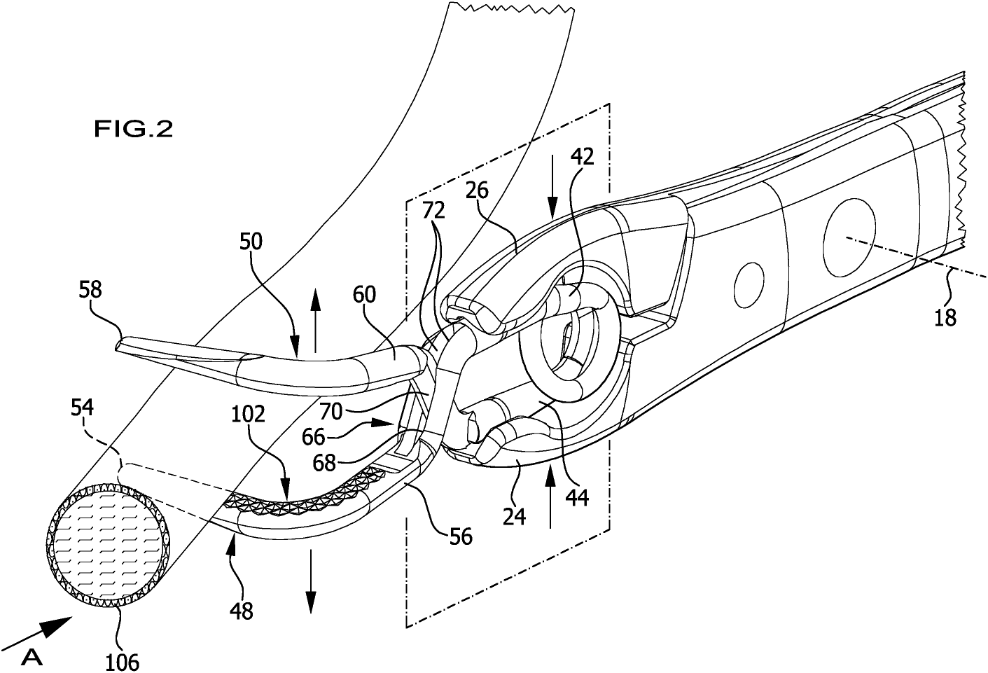

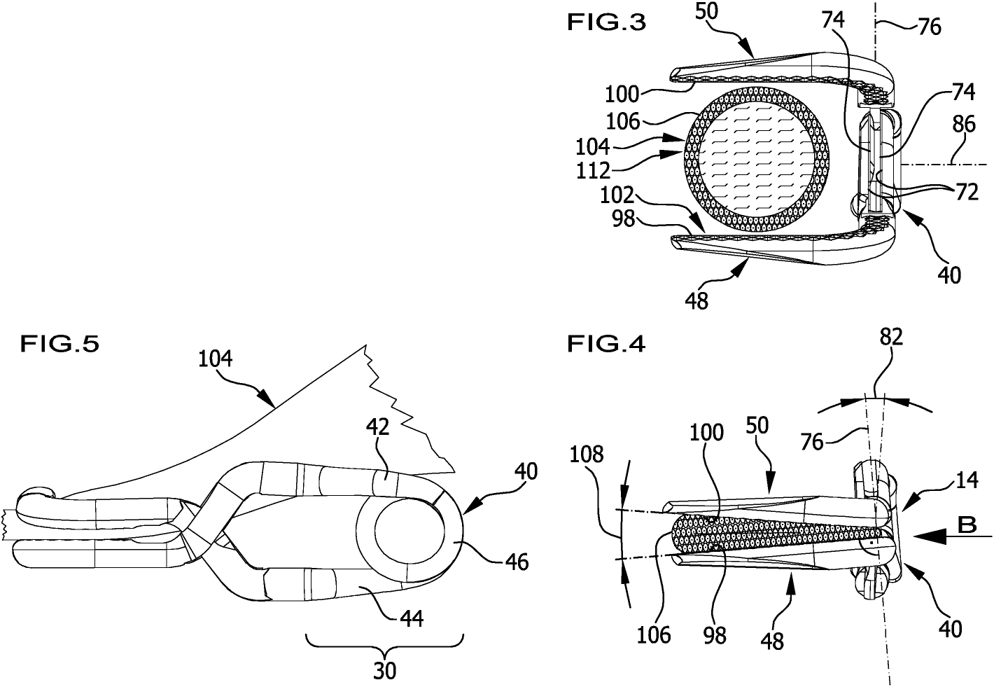

The clamping arms 48 and 50 can be moved away from one another against the action of the biasing element 40 from the basic position into an opening position. In FIGS. 2 and 3, the embodiment of the clip 14 is schematically depicted in an opening position.

In the embodiment of the clip 14 shown in FIGS. 1 to 5, the first clamping arm 48 commencing from the first clamping arm end 56 and the second clamping arm 50 commencing from the second clamping arm end 60 are of curved or angled configuration.

Two female delimiting surfaces 72 facing toward one another are formed at the connection perforation 68. Thus, the two female delimiting surfaces 72 laterally delimit the connection perforation 68. Two male delimiting surfaces 74 facing away from one another and toward the female delimiting surfaces 72 are formed on the connection portion 70.

The connection perforation 68 is of elongated hole-like configuration and defines a connection plane 76. The two female delimiting surfaces 72 extend in parallel to one another and in parallel to the connection plane 76.

In the embodiment of the medical clip 14 as schematically depicted in FIGS. 1 to 5, the male delimiting surfaces 74, which are arranged in the basic position with little play between the female delimiting surfaces 72, are each provided with a male torsional bevel 78. The male delimiting surfaces 74 extend in parallel to one another. Likewise, the male torsional bevels 78 extend in parallel to one another.

The two male delimiting surfaces 74 in the basic position extend in parallel or substantially in parallel to the connection plane 76.

As can be clearly seen, in particular, in FIGS. 8 and 9, no more than about 50% of the two male delimiting surfaces 74 are removed by the male torsional bevels 78.

The male torsional bevels 78 define planar bevel surfaces 80. These are configured extending on both sides without edges into the delimiting surfaces 74.

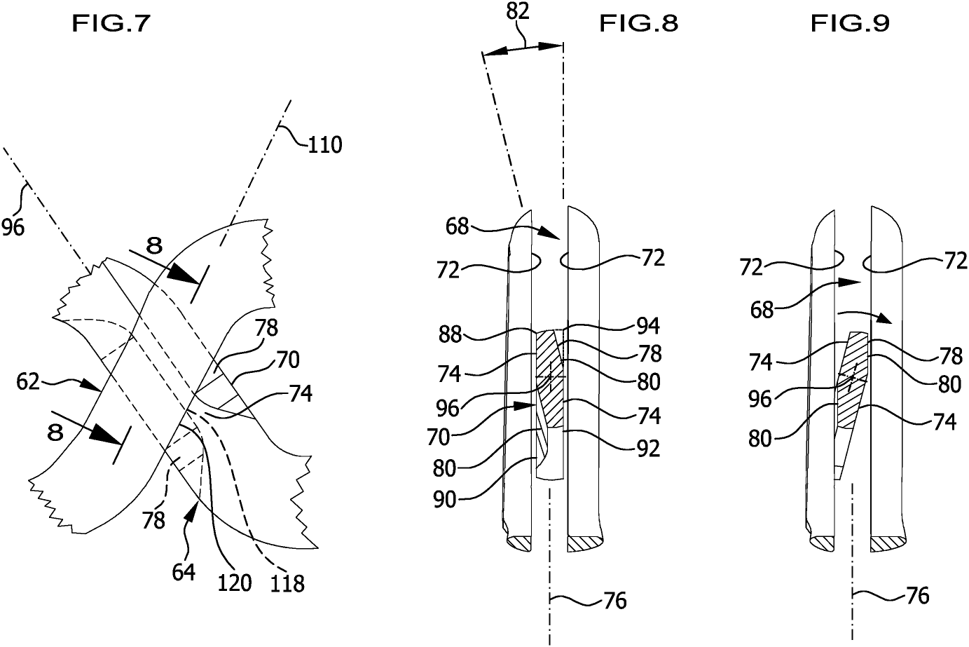

The male delimiting surfaces 74 and the associated bevel surfaces 80 of the male torsional bevels 78 enclose between them a male bevel angle 82. Said angle has a value in a range of about 5° to about 25°. In the embodiment depicted in FIGS. 7 to 9, the male bevel angle 82 is in a range of about 10° to about 18°. About 15° are shown.

The first clamping arm 48 and the second clamping arm 50 are curved out of the connection plane 76 or are angled toward it in such a way that the free ends 54 and 58 point in a direction that in the basic position runs transversely, perpendicularly in the embodiment depicted in the Figures, to the connection plane 76.

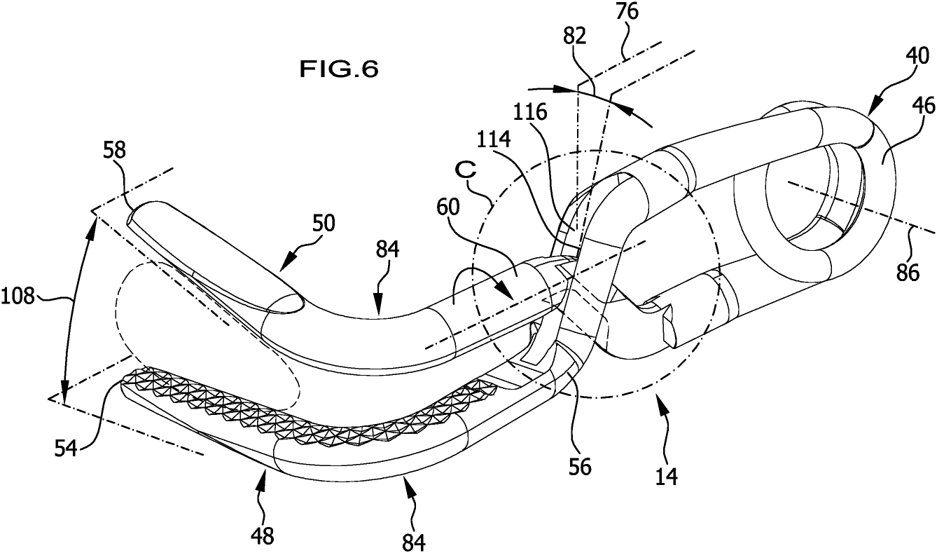

FIG. 6 illustrates schematically that the first clamping arm 48 and the second clamping arm 50 each have a bend 84, which in each case is arranged or formed between a free end 54 and 58 and the first and second clamping arm ends 56 and 60.

In the basic position, as schematically depicted in FIG. 1, the first clamping arm 48 and the second clamping arm 50 abut against one another. Thus, they extend in parallel to one another over their entire length.

The first clamping arm 48 and the second clamping arm 50 can be transferred, namely pivoted about a pivot axis 86, from the basic position depicted schematically in FIG. 1 into the application position depicted schematically in FIG. 2, in which the clamping arms 48 and 50 are further apart from one another relative to the basic position. The pivot axis 86 is defined substantially by a longitudinal axis of the coil spring 46, which extends transversely, in particular perpendicularly in the basic position, relative to the connection plane 76.

The connection portion 70 defines four connection portion edges 88, 90, 92, and 94. The two connection portion edges 90 and 94 that are initially present, for example, in a blank, which are rotated against one another by 180° relative to a second longitudinal axis 96 defined by the second connecting portion 64, are beveled by the two male torsional bevels 78, i.e. are no longer present.

When the medical clip 14 is accommodated in the receptacle 28 of the application instrument 12, by moving proximal ends of the branches 20 and 22 in the direction of the arrows 36 and 38 toward one another, the tool elements 24 and 26 can also be moved toward one another in order to move portions of the connecting portions 62 and 64 that abut against the tool elements 24 and 26 toward one another against the action of the biasing element 40. The clamping arms 48 and 50, the clamping surfaces 98 and 100 of which abut against one another in the basic position, hereby move away from one another. As schematically depicted in the embodiment of FIGS. 1 to 9, the clamping surfaces 98 and 100 may optionally be provided with a macroscopic surface structure 102 in order to reduce the risk of a hollow organ 104 slipping off when being accommodated between the clamping arms 48 and 50.

The opened clip 14 can be slid, as schematically shown in FIGS. 2 and 3, over the hollow organ 104, which is schematically depicted in the form of a blood vessel 106. When the actuating force on the proximal ends of the branches 20 and 22 is reduced, the leaf spring elements 32 and 34 push the branches 20 and 22 apart and thus the tool elements 24 and 26 move away from one another. The clip 14 is forced from the application position back into the basic position by the biasing element 40.

Due to the hollow organ 104 accommodated between the clamping surfaces 98 and 100, the clip 14 no longer closes completely. This means that in the clamping position, which is shown schematically in FIGS. 4, 6 and 9, the clamping surfaces 98 and 100 do not abut against one another, but remain slightly open.

Due to the curvature of the clamping arms 48 and 50, a torsional force acts on them, which is exerted by the hollow organ 104. This leads to a torsion of the clamping arms 48 and 50, in particular in the region of the box lock 66, and is shown in particular in a spreading of the clamping arms 48 and 50 to their free ends 54 and 58 such that the clamping surfaces 98 and 100 do not extend in parallel to one another as in the basic position, but instead enclose an opening angle 108 between them. The opening angle 108 is, in particular, smaller than the male bevel angle 82, but may also have approximately its value or be somewhat larger.

The particular design of the box lock 66 with the male torsional bevels 78 on the male delimiting surfaces 74 makes it possible to guide and to delimit a torsional movement of the connecting portions 62 and 64 relative to one another about a first longitudinal axis 110 of the first connecting portion 62 and the second longitudinal axis 96 of the second connecting portion 64 upon closing the clamping arms 48 and 50 when an object to be clamped 112, for example the hollow organ 104, is accommodated between then. In contrast to conventional clips, in the case of such a torsion of the connecting portions 62 and 64, the connection portion edges 90 and 94 do not abut in a point contact against connection perforation edges 114, 116, 118, and 120 of the connection perforation 68, namely concretely against the connection perforation edges 116 and 120, but instead a line contact is created between the male torsional bevels 78 and the bevel surfaces 80 defined thereby with the connection perforation edges 116 and 120. In this way, friction in the region of the box lock of angled or curved clips 14 can be reduced.

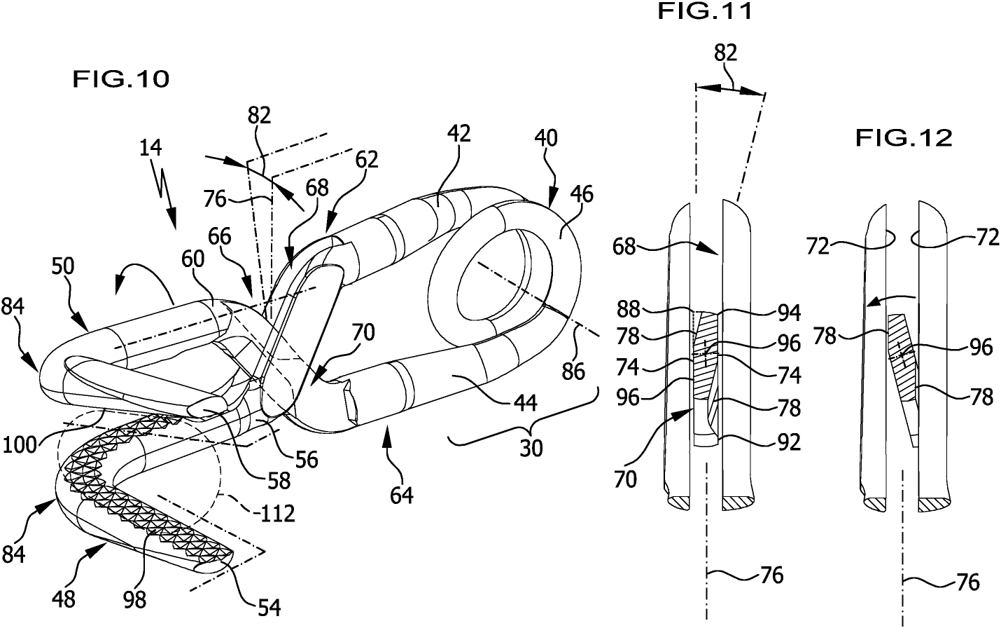

Schematically depicted in FIGS. 10 to 12 is a further embodiment of a medical clip 14. It is similar in its structure to the medical clip of FIGS. 6 to 9. It differs substantially in that the free ends 54 and 58 of the clamping arms 48 and 50 are bent in opposite directions relative to the connection plane 76, thus they point in opposite directions compared to the free ends 54 and 58 of the clamping arms 48 and 50 from the embodiment of the clip 14 shown in FIGS. 6 to 9.

The reference numerals used in FIGS. 10 to 12 correspond to the reference numerals used in connection with the embodiment of the clip 14 according to FIGS. 6 to 9.

If one compares FIGS. 6 and 10, one can see that the two clips 14 shown in these Figures are of mirror symmetrical configuration relative to one another. This ultimately does not change their function, but requires the formation of the male torsional bevels 78 on other connection portion edges of the connection portion 70, namely on the connection portion edges 88 and 92. The reason for this is that an object to be clamped 112 that is accommodated between the clamping surfaces 98 and 100 in the embodiment depicted in FIG. 10 leads to a torsional movement of the connection portion 70 about the second longitudinal axis 96, which is directed oppositely to the torsional movement of the connection portion 70 in the embodiment of FIGS. 6 to 9.

The two embodiments of the clips 14 according to FIGS. 6 to 9 on the one hand and according to FIGS. 10 to 12 on the other hand show, by way of example, that the male torsional bevels 78 are arranged in such a way that they enable a torsion of the connecting portions 62 and 64 relative to one another by the male bevel angle 82 by beveling the male delimiting surfaces 74 or the connection portion edges that otherwise lead to jamming in the case of conventional clips, namely in such a way that the torsion of the connecting portions 62 and 64 relative to one another is made possible in order to enable a line contact between the connecting portions 62 and 64 instead of a point contact.

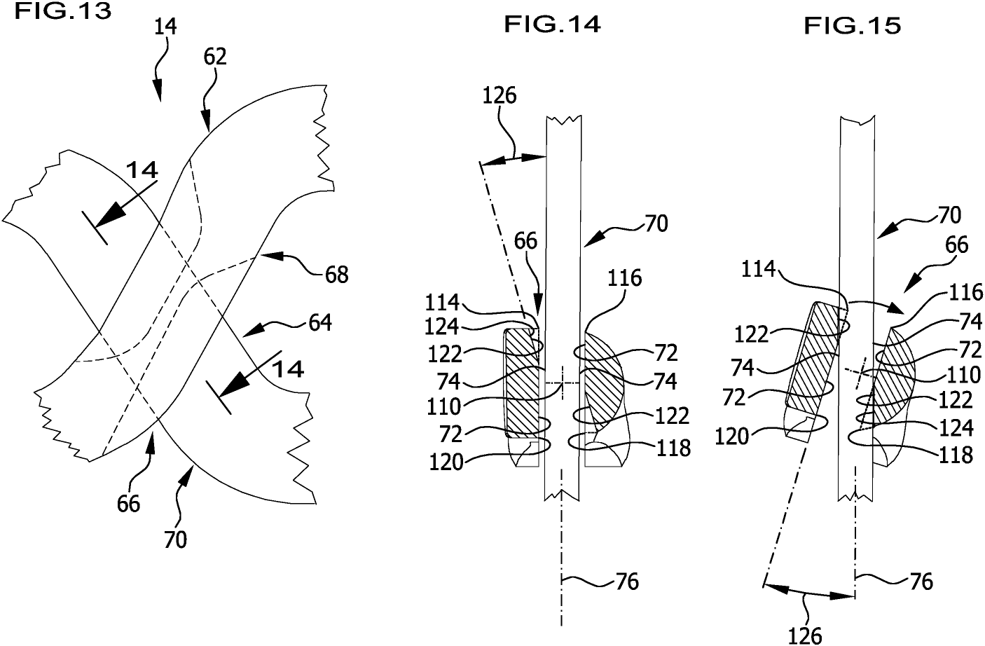

A further embodiment of a medical clip 10 is schematically depicted in part in FIGS. 13 to 15. This embodiment differs from the embodiment of FIGS. 6 to 9 only in the design of the box lock 66. With regard to all further features of the clip 14, reference is therefore made to the above description in connection with FIGS. 1 to 9. Therefore, identical reference numerals are also used to designate identical or equivalent elements and components.

In the embodiment of FIGS. 13 to 15, only the two female delimiting surfaces 72 are each provided with a female torsional bevel 122. These two female torsional bevels 122 extend in parallel to one another.

Without the female torsional bevels 122, four connection perforation edges 114, 116, 118 and 120 adjoining the female delimiting surfaces 72 would be defined at the connection perforation 68. In the depicted embodiment of FIGS. 13 to 15, the connection region perforation edges 114 and 118 are beveled by the female torsional bevels 122. These are rotated against one another by 180° relative to the first longitudinal axis 110 defined by the first connecting portion 62.

The female delimiting surfaces 72 and female bevel surfaces 124, which are defined by the female torsional bevels 122, enclose a female bevel angle 126, which has a value in a range of about 5° to about 25°. In one embodiment, the value is in a range of about 10° to about 18°. In the embodiment depicted in the Figures, the female bevel angle 126 is about 15°.

The female torsional bevels 122 are dimensioned such that at most about 50% of the two female delimiting surfaces 72 facing toward one another are removed by said bevels.

The female bevel surfaces 124 described are of planar configuration. They extend without edges into the female delimiting surfaces 72.

The embodiment of the clip 14 according to FIGS. 13 to 15 corresponds in its functioning to the embodiment of the clip 14, as it has been explained in detail in conjunction with FIGS. 10 to 12. By applying the female torsional bevels 122 in the manner described, a torsion of the connecting portions 62 and 64 relative to one another about the first longitudinal axis 110 is made possible, so that instead of a point contact between adjoining edges of the connection portion 70 and the connection perforation 68, a line contact is formed between two of the connection perforation edges 88, 90, 92 and 94 and the two female bevel surfaces 124 of the female torsional bevels 122.

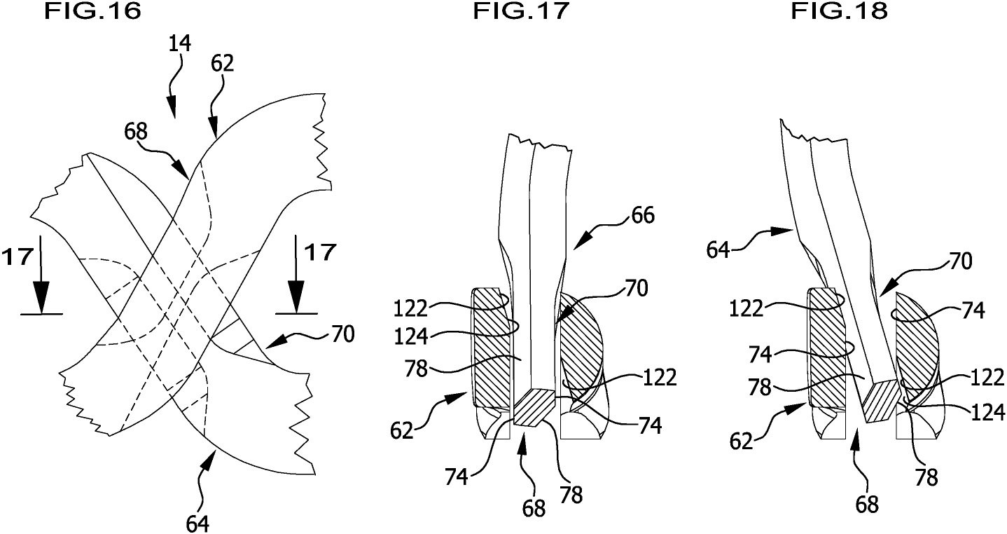

A further embodiment of a medical clip 14 is schematically depicted in part in FIGS. 16 to 18. The structure of the medical clip 14 of FIGS. 16 to 18 corresponds to the clip 14 of FIGS. 10 to 12, but differs from it in the design of the box lock 66.

In the embodiment of FIGS. 16 to 18, the connection portion 70 is configured corresponding to the connection portion 70 of the embodiment of FIGS. 10 to 12. In this embodiment, the connection perforation 68 is configured corresponding to the connection perforation 68 according to the embodiment of FIGS. 13 to 15. This enables not only a torsion about one of the two longitudinal axes 110 and 96, but about both longitudinal axes 110 and 96 simultaneously. By beveling both the female delimiting surfaces 72 and the male delimiting surfaces 74, the clamping arms 48 and 50 can torsion freely. A collision of the connecting portions 62 and 64 on both sides in the region of the box lock 66 is thus excluded. However, it is also necessary for the clip 14 to be oriented much more toward the tip, i.e. toward the free ends 54 and 58 of the clamping arms 48 and 50, in order to compensate for such a torsion. However, such an orientation results in a significant loss of the desired opening width of the clip 14.

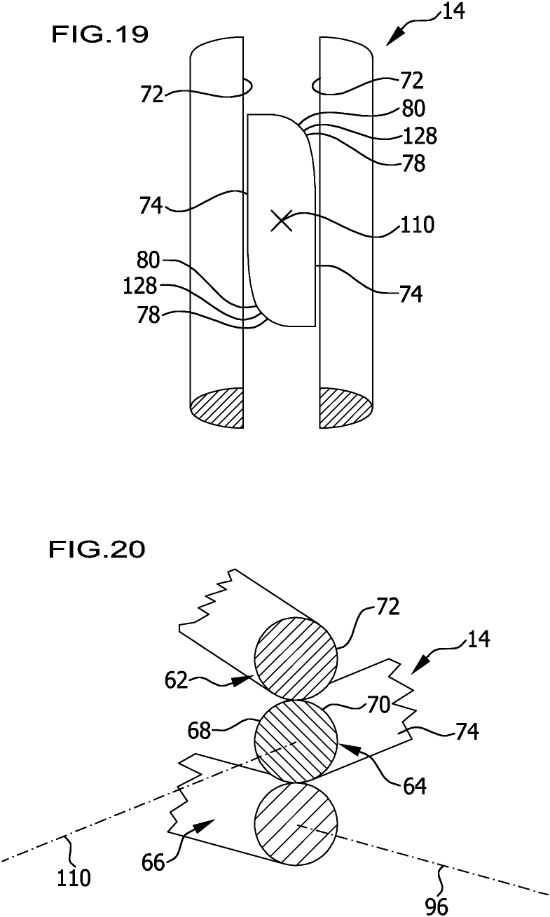

A further embodiment of a medical clip 14 is schematically depicted in FIG. 19. It is very similar to the embodiment of FIGS. 7 to 9 and differs from this only in the design of the male bevel surfaces 80. Unlike in the embodiment of FIGS. 7 to 9, these are not flat, but curved.

The curved bevel surfaces 80 define intersection lines 128 with an intersection plane at the second connecting portion 64 transverse to the male delimiting surfaces 74. The intersection lines 128 have an increasing curvature commencing from the beveled delimiting surfaces 74. In alternative embodiments that are not depicted, the curvature may also be constant.

In the embodiment depicted in FIG. 19, the intersection lines 128 of the male torsional bevels 78 define a section of an ellipse.

A further embodiment of a medical clip 14 is depicted in sections in FIG. 20. It corresponds in its structure with the clip schematically shown in FIGS. 1 to 9, but differs in the design of the box lock 66.

In this embodiment, the male delimiting surfaces 74 and the female delimiting surfaces 72 each define sections of outer surfaces of a straight circular cylinder. The longitudinal axes 110 and 96 of the connecting portions 62 and 64 each define circular cylinder longitudinal axes of the respective outer surfaces. In the embodiment shown in FIG. 20, all contact edges of the two connecting portions 62 and 64 relevant for a torsion thereof are beveled in the region of the box lock 66, namely provided with curved bevel surfaces. A pivoting, i.e. in particular a torsion of the clamping arms 48 and 50 relative to one another, does not cause a reduction of a play between the connection opening 68 and the connection portion 70 in that case of this design.

In the embodiment depicted in FIG. 20, outer surface radii of the male delimiting surfaces and the female delimiting surfaces are identical.

The connecting portions 62 and 64 of the embodiments of medical clips 14 described above, namely in particular their connection perforations 68 and/or connection portions 70, are optionally formed by milling, by electrochemical metal erosion, by a generative manufacturing process, in particular 3D printing, or by sink erosion.

In further embodiments, which correspond in their structure to the embodiments of the medical clips 14 described above, the male and/or female delimiting surfaces 72, 74 and the male and/or female torsional bevels 78, 122 are provided with a friction-reducing coating.

In the embodiments of medical clips 14 described above, the first clamping arm 48 and/or the second clamping arm 50 and/or the biasing element 40 are formed by a generative manufacturing process. In particular, in the embodiments described, the entire medical clip 14 is formed by a generative manufacturing process.

The medical clips described above are configured, in particular, in such a way that the first clamping arm 48 and the second clamping arm 50 are biased against one another in the basic position.

The embodiments of medical clips 14 described above are made of a biocompatible material.

The described and illustrated embodiments of medical clips are, in particular, formed in one piece. In this case, they are made of only one single biocompatible material.

In the embodiments described, the biocompatible material is optionally a metal, for example titanium, or a plastic.

In a further embodiment of a medical clip not shown in the Figures, a pre-twisted connection in the form of a box lock is provided. In this embodiment, the female and male delimiting surfaces are beveled neither at the connection perforation nor at the connection portion. Thus, no torsional bevels are provided. The pre-twisting of the connection is set in this embodiment such that an angle range of the pre-twisting corresponds to an angle range of the torsional bevels provided in the embodiments of clips described above. As a result of a pre-twisting of the connection set in this way, said pre-twisting is cancelled out at the measuring point, i.e. when the clip is opened slightly for determining the spring force, due to the torsion acting in opposite direction.

The targeted application of torsional bevels 78 and/or 122 described above in conjunction with various embodiments of medical clips 14 constitutes an effective means to reduce friction occurring in the region of the box lock 66. As described, this is particularly advantageous, in particular, in aneurysm clips 16 that have clamping arms 48 and 50 that are curved or are angled once or multiple times, also referred to as laterally deflected jaw parts, which absorb a torsional moment when placed against an object to be clamped 112. By reducing the friction due to the modified design of the box lock 66, there is a positive effect on a scattering of measured values in determining the spring force, i.e. the force exerted by the biasing element 40, so that waste in the manufacture of such clips can be reduced. In other words, due to the change in the shape of the box lock 66, it is possible to reduce the number of medical clips 14 whose clamping force or spring force is not in the range of the tolerance specified by the test standard.

What is claimed is:

1. A medical clip comprising:
a first clamping arm;
a second clamping arm;
a biasing element with a first end and a second end; and
a box lock,
the first clamping arm having a first clamping arm end connected to the first end of the biasing element by way of a first connecting portion,
the second clamping arm having a second clamping arm end connected to the second end of the biasing element by way of a second connecting portion,
the box lock having a connection perforation arranged or formed on the first connecting portion and a connection portion comprised by the second connecting portion and passing through the connection perforation,
the first clamping arm and the second clamping arm being maximally proximate to one another in a basic position of the medical clip and movable away from one another against an action of the biasing element from the basic position into an opening position,
the first clamping arm, commencing from the first clamping arm end, and the second clamping arm, commencing from the second clamping arm end, each having a curved or angled configuration in a direction toward free ends thereof,
two female delimiting surfaces facing toward one another being formed at the connection perforation, and
two male delimiting surfaces facing away from one another and toward the female delimiting surfaces being formed on the connection portion,
at least one of:
the female delimiting surfaces each being provided with a female torsional bevel; and
the male delimiting surfaces each being provided with a male torsional bevel.

2. The medical clip according to claim 1, wherein at least one of:
the female torsional bevels extend in parallel to one another; and
the male torsional bevels extend in parallel to one another.

3. The medical clip according to claim 1, wherein only the female delimiting surfaces are provided with a female torsional bevel.

4. The medical clip according to claim 1, wherein only the male delimiting surfaces are provided with a male torsional bevel.

5. The medical clip according to claim 1, wherein at least one of:
a) the male delimiting surfaces extend in parallel to one another, and
b) the connection portion is of elongated cuboidal configuration.

6. The medical clip according to claim 1, wherein the connection perforation has an elongated hole-like configuration and defines a connection plane, and wherein the female delimiting surfaces extend in parallel to the connection plane.

7. The medical clip according to claim 6, wherein at least one of:
a) the male delimiting surfaces in the basic position extend in parallel or substantially in parallel to the connection plane; and
b) the first clamping arm and the second clamping arm are curved out of the connection plane or are angled relative to the connection plane in such a way that free ends thereof point in a direction that runs transversely to the connection plane.

8. The medical clip according to claim 1, wherein at least one of:
a) the first clamping arm and the second clamping arm are continuously curved; and
b) the first clamping arm and the second clamping arm each have at least one bend, and wherein the at least one bend in each case is arranged or formed between a free end and the first and second clamping arm ends, and
c) the first clamping arm and the second clamping arm in the basic position extend in parallel to one another over their entire length.

9. The medical clip according to claim 1, wherein the first clamping arm and the second clamping arm are pivotable relative to one another about a pivot axis from the basic position into an abutment position in which the clamping arms are further apart from one another relative to the basic position.

10. The medical clip according to claim 1, wherein at least one of:
a) between about 40% and about 60% of the female delimiting surfaces are removed by the female torsional bevels, and
b) between about 40% and about 60% of the male delimiting surfaces are removed by the male torsional bevels.

11. The medical clip according to claim 1, wherein at least one of the female torsional bevels and the male torsional bevels define planar bevel surfaces.

12. The medical clip according to claim 1, wherein at least one of:
a) the female delimiting surfaces and female bevel surfaces, which are defined by the female torsional bevels, enclose a female bevel angle and wherein the female bevel angle has a value in a range of about 5° to about 25°; and
b) the male delimiting surfaces and male bevel surfaces, which are defined by the male torsional bevels, enclose a male bevel angle and wherein the male bevel angle has a value in a range of about 5° to about 25°.

13. The medical clip according to claim 1, wherein the female torsional bevels and the male torsional bevels define curved bevel surfaces.

14. The medical clip according to claim 13, wherein the curved bevel surfaces define intersection lines with an intersection plane at the respective connecting portion transverse to the delimiting surfaces thereof, and wherein the intersection lines have a constant or increasing curvature commencing from the beveled delimiting surface.

15. The medical clip according to claim 14, wherein the intersection lines of the torsional bevels define a section of an ellipse.

16. The medical clip according to claim 1, wherein at least one of:
a) the connection perforation defines four connection perforation edges adjoining the female delimiting surfaces and wherein two connection perforation edges that are rotated against one another by 180° relative to a first longitudinal axis defined by the first connecting portion are beveled by the female torsional bevels; and
b) the connection portion defines four connection portion edges and wherein two connection portion edges that are rotated against one another by 180° relative to a second longitudinal axis defined by the second connecting portion are beveled by the male torsional bevels.

17. The medical clip according to claim 1, wherein at least one of the female torsional bevels and the male torsional bevels are configured to guide and to delimit a torsional movement of the connecting portions relative to one another about longitudinal axes of the respective connecting portions upon closing the clamping arms when an object to be clamped is accommodated between them.

18. The medical clip according to claim 1, wherein the male delimiting surfaces and the female delimiting surfaces each define sections of outer surfaces of a straight circular cylinder and wherein connecting portion longitudinal axes of the connecting portions each define circular cylinder longitudinal axes of the respective outer surfaces.

19. The medical clip according to claim 18, wherein outer surface radii of the male delimiting surfaces and of the female delimiting surfaces are identical or substantially identical.

20. The medical clip according to claim 1, wherein at least one of:
a) the connecting portions are formed by milling, by electrochemical metal erosion, by a generative manufacturing process, or by sink erosion; and
b) at least one of the male delimiting surfaces and female delimiting surfaces and the male torsional bevels and the female torsional bevels are provided with a friction-reducing coating; and
c) at least one of the first clamping arm and the second clamping arm and the biasing element are formed by a generative manufacturing process; and
d) the first clamping arm and the second clamping arm are biased against one another in the basic position; and
e) the biasing element is configured in the form of a coil spring with at least one complete winding; and
f) the clip is made of at least one biocompatible material; and
g) the clip is configured in the form of an aneurysm clip.
